SEQUENCE LISTING (4 sequences)

SEQ ID NO: 1 - DNA, 1596 bp, Klebsiella pneumoniae
gagcaccgccgccgcaaggaatggtgcatgctgaggtgtctcacaagtgccgtgcagtcccgcccccacttgcttctctttgtgtgtagtgtacgtacattatcgagaccgttgttcccgcccacctcgatccggcatgctgaggtgtctcacaagtgccgtgcagtcccgcccccacttgcttctctttgtgtgtagtgtacgtacattatcgagaccgttgttcccgcccacctcgatccggcatgctgaggtgtctcacaagtgccgtgcagtcccgcccccacttgcttctctttgtgtgtagtgtacgtacattatcgagaccgttgttcccgcccacctcgatccggcatgctgaggtgtctcacaagtgccgtgcagtcccgcccccacttgcttctctttgtgtgtagtgtacgtacattatcgagaccgttgttcccgcccacctcgatccggcatgcactgatcacgggcaaaagtgcgtatatatacaagagcgtttgccagccacagattttcactccacacaccacatcacacatacaaccacacacatccacaatgaaaaagcctgaactcaccgcgacgagcgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcg

SEQ ID NO: 2 - DNA, 3279 bp, Saccharomyces cerevisiae
atgctttttgataacaaaaatcgcggtgctttaaactcactgaacacaccagatattgcttctttatcaatatcatccatgtcggactatcacgtgtttgattttcccggtaaggacctgcagagagaggaagtgatagatttgctagatcagcaagggtttattcccgacgatttgatcgaacaagaagtagattggttttataactcattgggtattgacgatttgttcttctcgagagaatctccccaattaatctcgaatatcatacattctttgtatgcttcaaagctagatttctttgcgaagtccaaattcaacggaattcagccaaggctattcagcattaaaaacaaaattataactaatgataatcatgccatctttatggaatctaatactggtgtcagcataagcgattctcagcaaaaaaactttaaatttgctagtgacgccgtcggaaacgatactttggagcatggtaaggataccatcaaaaaaaataggattgaaatggatgattcttgtccaccttatgaattagattccgaaattgatgaccttttcctggataacaagtctcaaaaaaactgcagattagtttctttttgggctccagaaagcgaattaaagctaacttttgtttatgagagtgtttaccctaatgatgatccagccggcgtagatatttcctctcaggatttgctgaaaggtgatattgaatcgattagtgataagaccatgtacaaagtttcgtcgaacgaaaataaaaaactatacggtctcttacttaagttggttaaagaaagagaaggtcctgtcattaagactactcgctccgtagaaaataaggatgaaattaggttattagtcgcttacaagcgattcaccactaagcgttattactctgctttgaactctttgttccactattacaagttgaaaccttctaagttctatttagagtcgtttaatgttaaggatgatgacatcattatcttttccgtttatttgaacgagaaccagcaattggaagatgttctacttcacgatgtggaggcagcattgaaacaggttgaaagagaagcttcattgctatacgctatcccaaacaattctttccatgaggtttaccagagacgtcaattctcgcccaaagaagctatatatgctcatattggtgctatattcattaaccattttgttaatcgtttaggctctgattatcaaaaccttttatctcaaatcaccattaagcgtaatgatactactcttttggagattgtagaaaacctaaaaagaaagttaagaaatgaaaccttaactcagcaaactattatcaacatcatgtcgaagcattacactataatttccaagttgtataaaaattttgctcaaattcactattatcataatagtactaaagatatggagaagacattatcttttcaaagactggaaaaagtggagccttttaagaatgaccaagagttcgaagcttacttgaataaattcattccaaatgattcacctgatttgttgatcctgaaaacactgaacatcttcaacaagtctattttgaagacaaatttctttattacaagaaaagtagcaatatcattcagattagatccttccctggtgatgacaaaattcgaatatccagagacaccctatggtatattttttgtcgttggtaatactttcaaagggttccatatcaggttcagagatatcgcaaggggcggtattcgtatagtctgttccaggaatcaggatatttatgatttgaattccaagaacgttattgatgagaactatcaattggcctctactcagcaacgtaaaaataaggatattccagagggtggctctaaaggtgtcatcttattgaacccaggattggtagaacatgaccagacatttgtcgccttttcccaatatgtggatgcaatgattgacattctaatcaacgatccattaaaggaaaactatgtcaaccttttaccaaaggaggaaatattattttttggcccagatgaaggaactgctggtttcgtggattgggcaactaaccatgctcgtgtgaggaactgcccatggtggaaatcatttttgactggaaaatccccatctttgggtggtattccccatgacgaatatggtatgacttctctgggtgttcgtgcttatgttaataaaatttacgaaactttaaacttgacaaattctactgtttacaaattccaaactggtggtccggatggtgatttgggatccaatgaaattcttttatcttcgccaaacgaatgttatttggcaattctggacggttcaggtgtcctgtgtgatcctaaaggtttagataaagatgaattatgccgcttggcacatgaaaggaaaatgatttccgatttcgacacttccaaattatcaaacaacggattttttgtttctgtggatgcaatggatatcatgctaccaaatggtacaattgtagctaacggcacaaccttcagaaacacctttcatactcaaattttcaaatttgtggatcatgtcgacatttttgttccatgcggtggtagaccaaactcaattactctaaataatctacattattttgttgacgaaaagactgggaaatgtaaaattccatatattgtggagggtgccaatctatttataacgcaacctgctaaaaatgctttggaggaacatggctgtattctgttcaaagatgcttctgcaaacaaaggtggtgtcacatcttcatcaatggaagtgttggcctcactagcgcttaacgataacgacttcgtgcacaaatttattggagatgttagtggtgagaggtctgcgttgtacaagtcgtacgttgtagaagtgcagtcaagaattcagaaaaatgctgaattagagtttggtcagttatggaatttgaatcaactaaatggaacccacatttcagaaatttcaaaccaattgtccttcactataaacaaattgaacgacgatctagttgcttctcaagagttgtggctcaatgatctaaaattaagaaactacctattgttggataaaataattccaaaaattctgattgatgttgctgggcctcagtccgtattggaaaacattccagagagctatttgaaagttcttctgtcgagttacttatcaagcacttttgtttaccagaacggtatcgatgttaacattggaaaattcttggaatttattggtgggttaaaaagagaagcggaggcaagtgcttga

SEQ ID NO: 3 - DNA, 19 bp, Artificial Sequence
  DNA Primer
cgtttgccagccacagatt

SEQ ID NO: 4 - DNA, 21 bp, Artificial Sequence
  DNA Primer
ttcaacctgtttcaatgctgc

(19) United States
(12) Patent Application Publication
(10) Pub. No.: US 2016/0138057 A1
(43) Pub. Date: May 19, 2016

(54) Methods for enhancing alpha-ketoglutarata production in Yarrowia lipolytica

(71) Applicants: Jian Chen, Wuxi (CN); Jingwen Zhou, Wuxi (CN); Hongwei Guo, Wuxi (CN); Weizhu Zeng, Wuxi (CN); Guocheng Du, Wuxi (CN)

(72) Inventors: Jian Chen, Wuxi (CN); Jingwen Zhou, Wuxi (CN); Hongwei Guo, Wuxi (CN); Weizhu Zeng, Wuxi (CN); Guocheng Du, Wuxi (CN)

(73) Assignee: Jiangnan University, Wuxi (CN)

(21) Appl. No.: 14/557,468

(22) Filed: Dec. 2, 2014

(30) Foreign Application Priority Data
Nov. 19, 2014 (CN) 201410666089.5

(51) Int. Cl. C12P 7/50; C12N 9/06

(52) U.S. Cl. CPC C12P 7/50; C12N 9/0016; C12Y 104/01002

(57) ABSTRACT

The present invention provides methods for enhancing α-KG production in Yarrowia lipolytica, relates to the field of metabolic engineering. This invention successfully overexpresses the glutamate dehydrogenase in wild type strain Y. lipolytica WSH-Z06 to construct the recombinant Y. lipolytica WSH-Z06 which regulates the glutamate catabolism to synthesis α-KG. L-methionine imine is added into the fermentation medium during the process to strengthen the supply of intracellular glutamate and inhibite the intracellular glutamine synthesis from glutamate metabolism and then enhance the accumunation of α-KG. Therefor, the present invention provides an effective method for enhancing the accumunation of α-KG through regulation of intracellular amino acid metabolism.

### CROSS-REFERENCES AND RELATED APPLICATIONS

This application claims the benefit of priority to Chinese Application No. 201410666089.5, entitled “Methods for enhancing alpha-ketoglutarata production in Yarrowia lipolytica”, filed Nov. 19, 2014, which is herein incorporated by reference in its entirety.

### BACKGROUND OF THE INVENTION

1. Field of the Invention

The present invention relates to the field of metabolic engineering, which relates to methods for enhancing alpha-ketoglutarata production in Yarrowia lipolytica, and more particularly relates to enhance alpha-ketoglutarata production in Yarrowia lipolytica by the regulation of intracellular amino acid metabolism.

2. Description of the Related Art

α-ketoglutarata(α-KG) is involved in many metabolic activities as one of the important intermediates of Krebs cycle (TCA) pathway in microbial cells. It is one of the key nodes in the citric acid cycle which involves in the synthesis of amino acids, proteins, vitamins and energy metabolism, and its accumulation in microbial cells is regulated by more factors compared with other intermediates of TCA. Therefore, to reveal the accumulation and regulation mechanism of α-KG in microbial cells has important significance, which would also guide the enhancement of accumulation of other products of TCA. α-KG is also an important chemical synthesis intermediates of the synthesis of amino acids, vitamins and other small molecules, which has important applications in the fields of pharmaceutical, organic synthesis, nutritional supplements, and so on. However, traditional chemical synthesis of α-KG has the following disadvantages: multiple steps, complex reaction process, the use of toxic compounds to human body such as cyanide. These prevent the applications of chemically synthesized α-KG in high value-added products, such as pharmaceutical and food. Microbial fermentation of α-KG production reduces the dependence on fossil energy supply, and the use of renewable biomass materials has many advantages, such as environmentally friendly and economically sustainable.

However, microbial production of α-KG also has shortcomings, such as low concentrations and low production intensity. The present invention provides a method to increase the production and accumulation of α-KG by modulating the synthesis of amino acids from α-KG.

### DETAILED DESCRIPTION

The goal of the present invention is to provide a recombinant Yarrowia lipolytica (Y. Lipolytica) enhancing the production of α-KG, which is constructed by overexpressing the glutamate dehydrogenase (GDH) in the starting strain Y. lipolytica to strengthen the enzyme activity of GDH and the supply of glutamate and accordingly enhance the accumulation of α-KG.

The gene encoding the GDH was from Saccharomyces cerevisiae (S. cerevisiae) in one of the embodiments of the present invention.

The nucleotide sequence of the gene encoding the GDH was SEQ ID NO.2 in one of the embodiments of the present invention.

Y. lipolytica is the starting strain of the recombinant Y. lipolytica and integrative expression vector p0 is the expression vector which containing a gene hph encoding a hygromycin phosphotransferase as screening marker in one of the embodiments of the present invention.

The starting strain was Y. lipolytica WSH-Z06 which preservation number is CCTCC NO: M20714 in one of the embodiments of the present invention.

The construction of plasmid P0 refers to: Swennen D, Paul MF, Vernis L, Beckerich JM, Fournier A, Gaillardin C. Secretion of active anti-Ras single-chain Fv antibody by the yeasts Y. lipolytica and Kluyveromyces lactis. Microbiology-Sgm, 2002. 148: 41-50.

The present invention also provides a method for enhancing the synthesis of α-KG in Y. lipolytica through the regulation of nitrogen metabolism in cells. Overexpressing of the GDH in the starting Y. lipolytica strengthened the activity of GDH and the supply of glutamate and accordingly enhanced the accumulation of α-KG.

L-methionine imine as glutamine synthetase inhibitor was added to reduce the metabolic breakdown of intracellular glutamate and enhance intracellular glutamate supply and α-KG accumulation in one of the embodiments of the present invention.

The present invention also provides a method for constructing the recombinant Y. lipolytica. The method comprises the following steps:
(1) Construction of integrative expression plasmid for target gene: the hph gene which encodes a hygromycin phosphotransferase was amplified and the nucleotide sequence of the hph gene is SEQ ID NO.1. The PCR products were digested with Stu I/Hind III and ligated into Stu I/Hind III-digested integrative expression vector p0 to create p0(hph) which containing a hygromycin phosphotransferase as screening marker.
(2) Construction of recombinant plasmid: the whole sequence of the open reading frame of GDH2 encoding an GDH was synthesized according to the sequences published on NCBI. The pruducts were digested with Sfi I/Not I and ligated into the plasmid p0(hph) to create a recombinant plasmid p0(hph)-GDH2.
(3) Transformation of p0(hph)-GDH2 into Y. lipolytica WSH-Z06: The recombinant plasmid p0(hph)-GDH2 was linearised with Avr II and then transformed into Y. lipolytica WSH-Z06 by electroporation. The genomes of transformants were extracted and verification primers VBF/V-GDH2 were used to screen the positive transformants to obtain recombinant Y. lipolytica strains named the Y. lipolytica-GDH2.

The present invention also provides a method for producing α-KG in recombinant Y. lipolytica. The activated recombinant Y. lipolytica strains were inoculated into the fermentation medium and incubated at 28-30° C. and 200-220 rpm for 144-168 hours.

In one of the embodiments of the present invention, the fermentation medium contained 100 g·L⁻¹ glycerol, 3 g·L⁻¹ (NH₄)₂SO₄, 3 g·L⁻¹ KH₂PO₄, 1.2 g·L⁻¹ MgSO₄·7H₂O, 0.5 g·L⁻¹ NaCl, 0.1 g·L⁻¹ K₂HPO₄, 2×10⁻⁷ g·L⁻¹ thiamine hydrochloride, and then adjusted to pH 5.0 and CaCO3 was added to it to 20 g·L⁻¹.

L-methionine imine was added into the fermentation medium in one of the embodiments of the present invention.

In one of the embodiments of the present invention, the fermentation medium contained 36.1 mg·L⁻¹ L-methionine imine, 100 g·L⁻¹ glycerol, 3 g·L⁻¹ (NH₄)₂SO₄, 3 g·L⁻¹ KH₂PO₄, 1.2 g·L⁻¹ MgSO₄·7H₂O, 0.5 g·L⁻¹ NaCl, 0.1 g·L⁻¹ K₂HPO₄, 2×10⁻⁷ g·L⁻¹ thiamine hydrochloride, and then adjusted to pH 5.0 and CaCO₃ was added to it to 20 g·L⁻¹.

In one of the embodiments of the present invention, the recombinant Y. lipolytica strain was incubated in seed medium at 28° C., 200 rpm for 16-18 hours and then inoculated into a 500 ml flask containing 50 ml fermentation medium with a inoculation volume of 10%, and incubated at 28° C. with a stirred revolutions of 200 rpm for 144-168 hours.

The GDH catalytic activity of the recombinant Y. lipolytica overexpressing the GDH rises to 8.62 U per mg protein, which is 7.2 times of the starting strain.

Adding of 0.2 mM glutamine synthetase inhibitor L-methionine imide during the fermentation of the recombinant strains reduces the breakdown of glutamate. The content of glutamate intracellular increases to 0.99 μmol per mg dry cells weight (DCW) with an increase of 86.3% and the accumulation of α-KG extracellular increases to 19.2 g·L⁻¹ with an increase of 32.4%.

The present invention enhances the metabolic flux from glutamate to α-KG by overexpression of GDH, and then strengthens the supply of intracellular glutamate by adding L-methionine imine in the fermentation process which significantly increases the extracellular accumulation of α-KG. Through the regulation of amino acid metabolism, the present invention reduces the synthesis of amino acids from α-KG in microbial cells, weakens the catabolism of α-KG and then enhances the accumulation of α-KG extracellular.

### BRIEF DESCRIPTION OF DRAWINGS

FIG. 1. Overexpression of GDH to increase intracellular catalytic activity of GDH; WSH-Z06 is the starting strain and GDH2 is the recombinant strain.

FIG. 2. Overexpression of GDH to enhance the synthesis of α-KG; WSH-Z06 is the starting strain and GDH2 is the recombinant strain.

FIG. 3. Adding of L-methionine imide to strengthen the supply of intracellular glutamate; WSH-Z06 is the starting strain and GDH2 is the recombinant strain.

FIG. 4. Changes of the content of extracellular external α-KG; WSH-Z06 is the starting strain and GDH2 is the recombinant strain.

### EXAMPLES

### Materials and Methods

YPD medium: 10 g·L⁻¹ yeast extract, 20 g·L⁻¹ peptone, 20 g·L⁻¹ dextrose.

Solid YPD medium: YPD medium with 20 g·L⁻¹ agar. Hygromycin B was added to a final concentration of 400 mg·L⁻¹ when screening the positive recombinant strain.

Seed medium: 20 g·L⁻¹ glucose, 10 g·L⁻¹ peptone, 0.5 g·L⁻¹ MgSO₄·7H₂O, 1.0 g·L⁻¹ KH₂PO₄, the pH was adjusted to 5.5 with dilute hydrochloric acid and then sterilized at 115° C. for 15 min. 20 g·L⁻¹ agar was added in solid medium.

Fermentation medium: 36.1 mg·L⁻¹ L-methionine imine, 100 g·L⁻¹ glycerol, 3 g·L⁻¹ (NH₄)₂SO₄, 3 g·L⁻¹ KH₂PO₄, 1.2 g·L⁻¹ MgSO₄·7H₂O, 0.5 g·L⁻¹ NaCl, 0.1 g·L⁻¹ K₂HPO₄, 2×10⁻⁷ g·L⁻¹ thiamine hydrochloride, pH 4.5, and then sterilized at 115° C. for 15 min. CaCO₃ sterilized at 121° C. for 30 min was added to 20 g·L⁻¹ before inoculation.

Y. lipolytica WSH-Z06 was obtained from China Center for Type Culture Collection with a preservation number CCTCC NO: M20714.

Determination of GDH catalytic activity and intracellular amino acid content: cells were collected by centrifugation in the exponential growth phase and washed by 0.9% saline, then suspended by 10 mL buffer (0.1 M KH₂PO₄-K₂HPO₄, 1 mM EDTA, 0.01 mM DTT, pH 7.5). Pickling glass beads was added for grinding for 5 min at 4° C., and then centrifuged at 13000×g for 10 min. The supernatant was used for the determination of catalytic activity of GDH and the intracellular amino acid content.

Quantification of the catalytic activity of GDH: A reaction mixture (6 mM NAD⁺, 100 mM glutamic acid, 160 mM glycine, 1.8 mM NaCl, 1.8 mM NaCl, pH=9.0) was added to 1.5 ml cell disruption supernatant to a total volume of 3 mL. The content of NADH was detected at 340 nm, 30° C. One unit of catalytic activity was defined as the amount of enzyme required for the generating of 1 μmol NADH per unit time.

Intracellular glutamate and glutamine was determinated by the following steps: 200 μl cell disruption supernatant and 800 μL 5% trichloroacetic acid was sequentially added to an 1 mL EP tube and standed for 5 min. 1 mL sample was filtered by 0.22 μm aqueous filter head and then centrifuged at 10000 rpm for 10 min. The amino acid content of the treated sample was measured by HPLC.

The HPLC conditions were as follows: pre-column derivatization of sample was carried out by o-xylene (OPA) and 9-Fluorenylmethyl Chloroformate. Mobile phase A was made by the following steps: 1000 mL water was added to 5.0 g sodium acetateanhydrous in a 1000 mL beaker and stirred until fully dissolved; Then 200 μL triethylaminewas add, and 5% acetic acid was dropwised to pH 7.20±0.05 while stirring; 5 mL tetrahydrofuran was added and mixed for later use. Mobile phase B was made by the following steps: 400 mL acetonitrile, 400 mL methanol was sequentially added to 5.0 g anhydrous sodium acetate in 1000 mL beaker and then ultra-pure water was added to dilute to 1000 mL. Stired until fully dissolved and adjusted the pH to 7.20±0.05. Mixed for later use. The column was ODS-2 Hypersil (250 mm×4.6 mm×5 μm), column temperature was 40° C. and the excitation wavelength was 338 nm of the UV detector. The elution procedure was shown in Table 1.

TABLE 1
The gradient elution of amino acid analysis

| Time (min) | A % | B % | Velocity of flow (ml/min) |
|---|---|---|---|
| 0 | 92 | 8 | 1 |
| 27.5 | 40 | 60 | 1 |
| 31.5 | 0 | 100 | 1.5 |
| 32 | 0 | 100 | 1.5 |
| 34 | 0 | 100 | 1 |
| 35.5 | 92 | 8 | 1 |

Extracellular α-KG was quantified by HPLC. Appropriate supernatant of fermentation broth centrifuged at 13000×g was diluted 50-fold with ultrapure water and filtrated by 0.22 μm filter for HPLC analysis. The column was Aminex HPX-87H ion exchange column and 5 mmol·L⁻¹ sulfuric acid solution (550 μL concentrated sulfuric acid volumed to 2 L) filtrated by 0.22 μm filter and degassed was used as mobile phase. The velocity of flow was 0.6 mL·min⁻¹ with a column temperature at 35° C. and a injection volume of 10 μL. The wavelength of UV detector was 210 nm.

The transformation of p0(hph)-GDH2 into Y. lipolytica WSH-Z06 was achieved by electroporation. Fresh Y. lipolytica WSH-Z06 single colony on YPD medium was transferred to YPD liquid medium and incubated at 28° C., 200 rpm overnight. Then the seeds were transferred to fresh YPD liquid medium with 10% inoculation and incubated at the same conditions to OD₆₀₀ 1.2. The cells were collected by centrifugation and treated with pH 7.5 buffer (8 mL 100 mmol·L⁻¹ LiAc, 10 mmol·L⁻¹ DTT, 0.6 mol·L⁻¹ sorbitol 10 mmol·L⁻¹ Tris-HCL) at 30° C. Then the cells were collected by centrifugation and washed by 5 mL ice-cold sorbitol for three times and suspended with 1 mol·L⁻¹ sorbitol to 10¹⁰ cells per mL. 1 μg pre-linearized integrative vector was added to the cell suspension and placed on ice for 5 min. The mixture was transferred to a prechilled 0.2 cm electroporation cuvette and shocked at 2.5 KV, 25 μF, 200 Ω. 1 mL ice-cold 1 mol·L⁻¹ sorbitol was added immediately and standed at room temperature for 1 hour. The 0.2 mL electric shocked products were applied to screening plates containing 400 mg·L⁻¹ hygromycin B and cultured at 28° C. for 48-72 hours.

TABLE 1
Verification primers for positive recombinant
strain.

| Primers | Sequences (from 5′ to 3′) |
|---|---|
| VBF | CGTTTGCCAGCCACAGATT |
| V-GDH2 | TTCAACCTGTTTCAATGCTGC |

### Example 1 Influence of Overexpression of GDH on the Growth of Cells

Construction of the recombinant strain Y. lipolytica-GDH2 was carried out by the following steps:
(1) Construction of integrative expression plasmid for target gene: the hph gene which encoded a hygromycin phosphotransferase was amplified and the nucleotide sequence of the hph gene is SEQ ID NO.1. The PCR products were digested with Stu I/Hind III and ligated into Stu I/Hind III-digested integrative expression vector p0 to create p0(hph) which containing a hygromycin phosphotransferase as screening marker.
(2) Construction of recombinant plasmid: the whole sequence of the open reading frame of GDH2 encoding an GDH was synthesized according to the sequence published on NCBI which was from S. cerevisiae with the GeneID:851311. The pruducts were digested with Sfi I/Not I and ligated into the plasmid p0(hph) to create a recombinant plasmid p0(hph)-GDH2.
(3) Transformation of p0(hph)-GDH2 into Y. lipolytica WSH-Z06: The recombinant plasmid p0(hph)-GDH2 was linearised with Avr II and then transformed into Y. lipolytica WSH-Z06 by electroporation. Transformants was selected on the screening plate which containing 400 mg·L⁻¹ hygromycin B. The genome of transformants was extracted and validated by primers VBF/V-GDH2 (As shown in Table 1) to screen the positive transformants to obtain recombinant Y. lipolytica strains named the Y. lipolytica-GDH2.

The construction of plasmid P0 refers to: Swennen D, Paul MF, Vernis L, Beckerich JM, Fournier A, Gaillardin C. Secretion of active anti-Ras single-chain Fv antibody by the yeasts Y. lipolytica and Kluyveromyces lactis. Microbiology-Sgm, 2002. 148: 41-50.

The wild type Y. lipolytica WSH-Z06 and the recombinant strain Y. lipolytica-GDH2 were incubated in 250 mL flasks with 20 mL YPD medium at 28° C., 200 rpm to exponential growth phase (about 20 hours) at the same time. Cells were centrifugated and washed by physiological saline for two times. The intracellar catalytic activities of GDH, the contents of glutamate and glutamine contents were quantified by the methods described above.

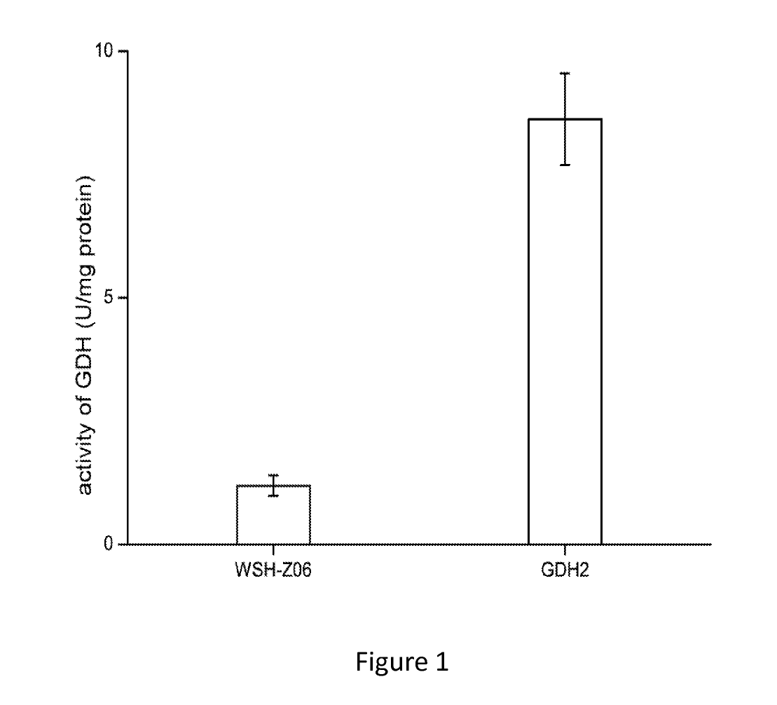

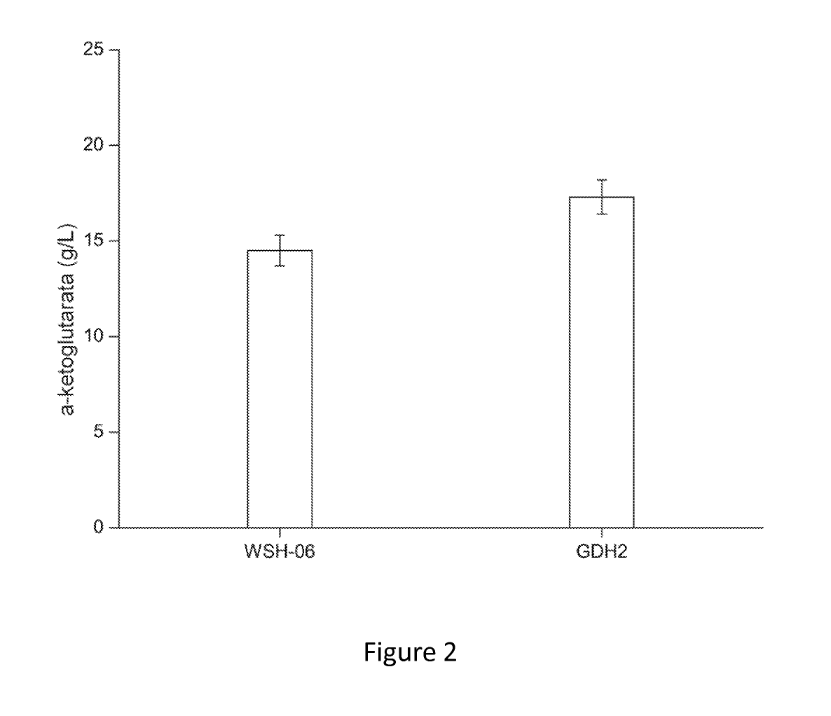

Compared to the wild type strain, the intracellar catalytic activity of GDH of the recombinant strain Y. lipolytica-GDH2 rose to 8.62±1.02 U per mg protein, which was 7.2 times of the wild type as shown in FIG. 1. And the α-KG content accumulated extracellular increased to 17.4 g·L⁻¹ from 14.5 g·L⁻¹ as shown in FIG. 2.

### Example 2 Enhancement of Glutamate Supply by Adding L-Methionine Imide

The wild type Y. lipolytica WSH-Z06 and the recombinant strain Y. lipolytica-GDH2 were incubated in 500 mL flasks with 50 mL fermentation medium at 28° C., 200 rpm for 96 hours at the same time. The fermentation broths were centrifugated to collect the cells. The glutamate contents were quantified by the methods described above.

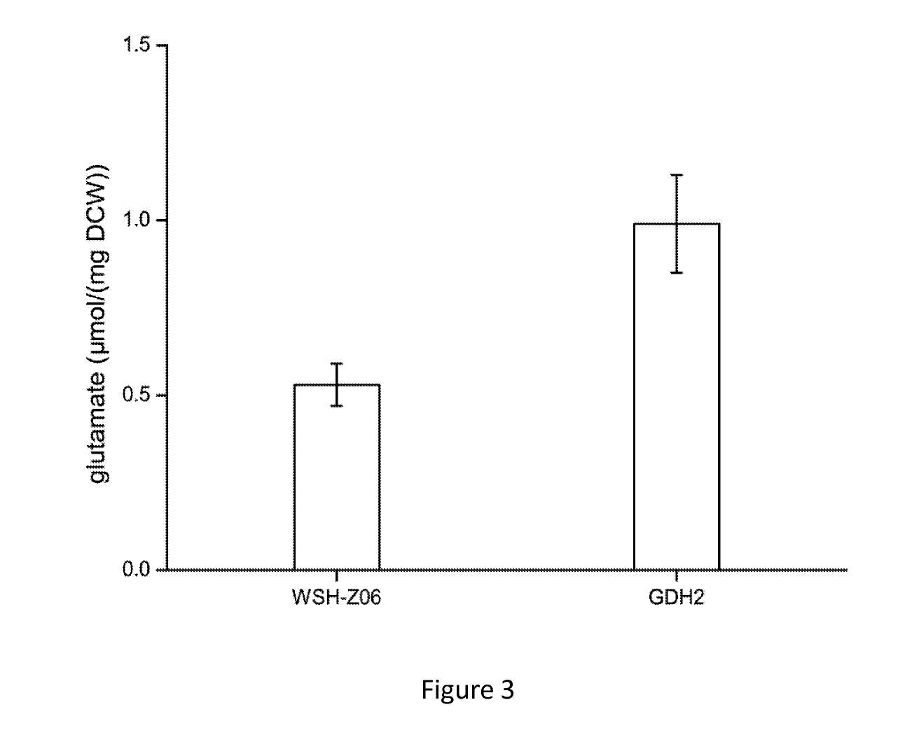

Compared to the wild type strain, the intracellar glutamate contents increased to 0.99 μmol per mg DCW from 0.53 μmol per mg DCW as shown in FIG. 3.

### Example 3 Enhancement of the Accumulation of α-KG by the Regulation of Intracellular Amino Acid Metabolism

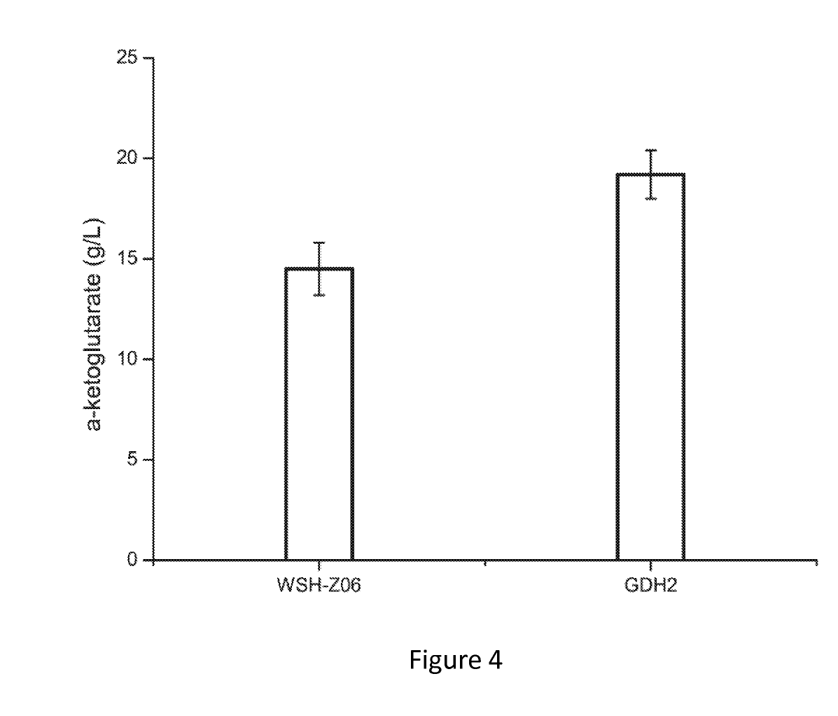

α-KG was produced as the method described above. The wild type Y. lipolytica WSH-Z06 and the recombinant strain Y. lipolytica-GDH2 were incubated in 500 mL flasks with 50 mL fermentation medium containing L-methionine imide at 28° C., 200 rpm for 144 hours at the same time to accumulate α-KG extracellular. Comparison indicated that enhancement of glutamate supplying and glutamate metabolized to α-KG significantly increased the catalytic activity of GDH of the recombinant strain Y. lipolytica-GDH2 and the α-KG accumulated extracellular increased to 19.2 g·L⁻¹ from 14.5 g·L⁻¹ as shown in FIG. 4.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A recombinant Yarrowia lipolytica (Y. lipolytica) which enhances the production of α-KG, wherein the recombinant strain overexpresses a glutamate dehydrogenase to enhance the supply of glutamate.

2. The recombinant Y. lipolytica of claim 1, wherein the glutamate dehydrogenase is from Saccharomyces cerevisiae.

3. The recombinant Y. lipolytica of claim 1, wherein the recombinant strain has an integrative expression vector p0 containing a hygromycin phosphotransferase as screening marker; and wherein the nucleotide sequence of glutamate dehydrogenase is set forth in SEQ ID NO:2.

4. The recombinant Y. lipolytica of claim 2, wherein the recombinant strain has an integrative expression vector p0 containing a hygromycin phosphotransferase as screening marker; and
wherein the nucleotide sequence of glutamate dehydrogenase is set forth in SEQ ID NO:2.

5. A method for enhancing the synthesis of α-KG in Y. lipolytica, comprising overexpressing a glutamate dehydrogenase to increase intracellular activity of glutamate dehydrogenase and the supply of glutamate, which leads to enhanced accumulation of α-KG.

6. The method of claim 5, further comprising adding L-methionine imine to a fermatation process of Y. lipolytica to reduce metabolic breakdown of intracellular glutamate.

7. A method for producing α-KG in the recombinant Y. lipolytica of claim 1, comprising inoculating the recombinant strain into a fermentation medium and incubating at 28-30° C., 200-220 rpm for 144-168 hours.

8. The method of claim 7, wherein the fermentation medium contains 100 g·L⁻¹ glycerol, 3 g·L⁻¹ (NH₄)₂SO₄, 3 g·L⁻¹ KH₂PO₄, 1.2 g·L⁻¹ MgSO₄·7H₂O, 0.5 g·L⁻¹ NaCl, 0.1 g·L⁻¹ K₂HPO₄, 2×10⁻⁷ g·L⁻¹ thiamine hydrochloride, and is adjusted to pH 5.0 by adding CaCO₃ 20 g·L⁻¹.

9. The method of claim 7, further comprising adding L-methionine imine into the fermentation medium.

10. The method of claim 7, wherein the fermentation medium contains 36.1 mg·L⁻¹ L-methionine imine, 100 g·L⁻¹ glycerol, 3 g·L⁻¹ (NH₄)₂SO₄, 3 g·L⁻¹ KH₂PO₄, 1.2 g·L⁻¹ MgSO₄·7H₂O, 0.5 g·L⁻¹ NaCl, 0.1 g·L⁻¹ K₂HPO₄, 2×10⁻⁷ g·L⁻¹ thiamine hydrochloride, and is adjusted to pH 5.0 by adding CaCO₃ 20 g·L⁻¹.
